SEQUENCE LISTING (28 sequences)

SEQ ID NO: 1 - protein, 801 aa, Zea mays
MALPSQAPSNSGDPLYPELWRACAGPLVTVPRVGDLVFYFPQGHIEQVEASMNQVAGNPMRLYDLPSKLLCRVLNVELKAETDTDEVYAQIMLMPEPEQNDVAAEKTSSGSAAPPRPAVRSFCKTLTASDTSTHGGFSVLRRHADECLPALDMSQSPPTQELVAKDLHGMEWRFRHIFRGQPRRHLLQSGWSVFVSSKRLVAGDAFIFLRGENGELRVGVRRAMRQLSNVPSSVISSQSMHLGVLATAWHAINTKSMFTVYYKPRTSPSEFIIPYDQYMESVKNNYSIGMRFRMRFEGEEAPEQRFTGTIVGCENLDPLWPDSSWRYLKVRWDEPSTIPRPDKVSPWKIEPASSPPVNPLPLSRGKRPRQNAPPPSPESSVLTKEGATKIDTDSAQTPHQNSVLQSQEQMSFRNNLTESTDSDSTVQKQMMWSPSPNGKVHTNFQPRPAMDNWMPLGRRETDFKDTRSAFKDARTASQSFGDTQGFFMQAYDDSRHRLSFNNQFQDQGSAHRFADPYFYMPQQPSLTVESGTRTQTANNDLRFWSERNSMYGNPSDQQQGFSFGQNTSSWLNQPLPQVEQSRVVRPHATVAPFDLEKTREGSGFKIFGFQVDTTNPSPVQLSSPLSAIREHVVQTRPSAPVNELQPVQIECLPEVSVSTAGTAAENIQQVQQSSKDIQSKSQGASTRSCTKVHKQGVALGRSVDLSKFTDYGELKAELDKMFEFEGELVSANRNWQIVYTDNEGDMMLVGDDPWEEFCNIVRKIYIYTKEEVQKMNSKSSVPRKEEPPAAGEGCAAAAANE

SEQ ID NO: 2 - DNA, 2406 bp, Zea mays
atggcgctgccgtcccaggccccgtctaactcaggggatccgctgtacccggagctctggcgcgcctgcgccggcccgctcgtcaccgtcccccgcgtcggcgacctcgtcttctacttcccccagggacacatcgagcaggtggaggcgtccatgaaccaggttgccgggaacccgatgcgtctctacgatctgccttccaagctgctctgccgcgtgctcaacgtcgagctgaaggcggagaccgacaccgacgaggtgtacgcgcagatcatgctcatgccggagcccgagcaaaacgatgtggctgccgagaagacgagctctgggtctgctgcgccaccgaggccagcggtcaggtcattctgcaagacgctcaccgcctccgacaccagcacacacggcggcttctctgtcctgcgccgccacgctgacgagtgcctccctgccctggatatgtcccagtcgcctcccactcaggagctggtggcgaaggatctgcatggcatggagtggcgattccgccacatcttccgcgggcaacccaggaggcatctccttcagagtggttggagtgtttttgtcagttccaaaaggcttgttgctggagacgccttcattttcctcagaggagaaaacggtgaacttcgagttggtgttaggcgcgctatgaggcagttgtctaatgtaccttcttcggttatatctagccaaagcatgcatctcggggttcttgcgactgcatggcacgctattaacacaaagtccatgttcactgtctactacaaacctaggacgagcccttcagagttcatcattccatatgatcaatatatggagtcagtaaaaaacaattattctattgggatgagattcaggatgaggtttgaaggggaagaggcccccgagcagaggtttactggtacaatagttggctgtgaaaatcttgacccactatggcctgattccagttggagatacttgaaggtgcgctgggatgagccttctactatcccgcggccagataaggtctctccctggaagatagagcctgcttcatcacctcctgttaatcctctccctctttctagaggaaaaagacctagacaaaatgcacctccaccttcccctgaatcatctgttctcacaaaagaaggtgcaaccaagattgatactgattctgctcaaacaccacatcaaaattcggtcttgcaaagtcaagagcagatgagctttaggaacaacctgactgaaagcaccgactctgattccactgttcagaagcagatgatgtggtccccatcacccaatgggaaagttcacactaattttcagccgagacctgctatggataactggatgccattgggaaggcgtgaaactgacttcaaggacacccgttctgccttcaaggatgcccgcaccgcttctcaatcgtttggagatacacaggggttctttatgcaagcttatgatgacagtcgtcaccgtctttctttcaacaatcagtttcaagatcagggttcagctcatcgctttgcagatccatacttctatatgcctcaacaaccttctctgacggttgaatcaggcacaaggacgcaaacagcgaacaatgacttgcgtttttggagtgagcggaattcgatgtatggtaatccaagtgaccaacagcagggtttcagctttgggcaaaacacatcaagttggttgaaccagccacttccccaggttgaacagtcacgagtggtcaggccgcatgcaacggttgctccatttgatttagagaagactagagaaggcagtgggttcaaaatatttggtttccaagttgatacaaccaatccatcacctgtccaattgagctctccattgtctgctatccgagagcatgtggtacaaactcgaccatcggcaccagtgaatgaattgcaacctgtgcaaattgagtgcttgcctgaggtgtccgtaagcacagctggaactgcagctgagaacattcagcaagtccagcaaagttcgaaagatattcaaagcaagtcccagggcgcttcaacaaggagctgtacaaaggttcataagcaaggagttgcgcttggccggtccgtggacctctctaaattcaccgactacggtgagctcaaagcggaactagacaagatgtttgagttcgagggcgaattggtatctgctaaccggaactggcagatcgtttataccgacaacgagggtgatatgatgcttgtcggagatgacccgtgggaggagttctgcaacatagtgcgcaagatctacatctacacgaaggaggaggtccagaagatgaactcgaaatcatccgtgccgaggaaggaggaacccccagcagcaggcgaaggctgcgccgccgccgccgcaaacgagtag

SEQ ID NO: 3 - protein, 808 aa, Oryza sativa
MPPAAMAPPPPPQGSSTGDPLYDELWHACAGPLVTVPRVGDLVFYFPQGHIEQVEASMNQVADSQMRLYDLPSKLLCRVLNVELKAEQDTDEVYAQVMLMPEPEQNEMAVEKTTPTSGPVQARPPVRSFCKTLTASDTSTHGGFSVLRRHADECLPPLDMTQSPPTQELVAKDLHSMDWRFRHIFRGQPRRHLLQSGWSVFVSSKRLVAGDAFIFLRGENGELRVGVRRAMRQLSNVPSSVISSQSMHLGVLATAWHAINTKSMFTVYYKPRTSPSEFIIPYDQYMESVKNNYSVGMRFRMRFEGEEAPEQRFTGTIIGSENLDPVWPESSWRSLKVRWDEPSTIPRPDRVSPWKIEPASSPPVNPLPLSRVKRPRPNAPPASPESPILTKEAATKVDTDPAQAQRSQNSTVLQGQEQMTLRSNLTESNDSDVTAHKPMMWSPSPNAAKAHPLTFQQRPPMDNWMQLGRRETDFKDVRSGSQSFGDSPGFFMQNFDEAPNRLTSFKNQFQDQGSARHFSDPYYYVSPQPSLTVESSTQMHTDSKELHFWNGQSTVYGNSRDRPQNFRFEQNSSSWLNQSFARPEQPRVIRPHASIAPVELEKTEGSGFKIFGFKVDTTNAPNNHLSSPMAATHEPMLQTPSSLNQLQPVQTDCIPEVSVSTAGTATENEKSGQQAQQSSKDVQSKTQVASTRSCTKVHKQGVALGRSVDLSKFSNYDELKAELDKMFEFDGELVSSNKNWQIVYTDNEGDMMLVGDDPWEEFCSIVRKIYIYTKEEVQKMNSKSNAPRKDDSSENEKGHLPMPNKSDN

SEQ ID NO: 4 - protein, 801 aa, Hordeum vulgare
MPPPAMATPQAPSAGDPLYDELWHACAGPLVTVPRVGDLVFYFPQGHIEQVEASMNQVAGNQMRLYDLPPKLLCRVINVELKAEADTDEVYAQVMLMPEPEQNEMAVDKSTSTTGATPPRPAVRSFCKTLTASDTSTHGGFSVLRRHADECLPPLDMTQSPPTQELVAKDLHGMDWRFRHIFRGQPRRHLLQSGWSVFVSSKRLVAGDAFIFLRGESGELRVGVRRAMRQLSNVPSSVISSHSMHLGVLATAWHAINTKSMFTVYYKPRTSPSEFIIPYDQYMESVKNNYSIGMRFRMRFEGEEAPEQRFTGTIVGSENLDQLWPESNWRSLKVRWDEPSTIPRPDRVSPWKIEPASSPPVNPLPLSRVKRPRPNVPPVSPESSVLTKEGATKIDMDSAQAQQRNQNSMVLQGQEHMTLRTNNLTGSNDSDATVQKPMMWSPSPNIGKNHASAFQQRPSMENWMQLGRCETAFKDASSGAQSFGDSQGFFMQTFDEAPNRHGSFKNQFQDHSSARHFSDPYTKMHTEANEFHFWNSQSTVYGNPRDQSQGFRFEEHPSNWLRQQQFSPVEQPRVIRPQASIAPVDLEKAREGSGFKIFGFKVDTTSAPSNHLSSTMAVIHEPVLQTQASASLTQLQHAHIDCIPELSVSTAGTTENEKSIQQAPNSSKDVQSKSHGASTRSCTKVHKQGVALGRSVDLSKFGDYDELTAELDRMFEFDGELMSSNRDWQIVYTDPEGDMMLVGDDPWEEFCSIVRKIFIYTKEEVQKMNSKSSTPRKEEGSADADGANEKAHLAASSHLDN

SEQ ID NO: 5 - protein, 704 aa, Sorghum bicolor
MPPAAAMALPSQAPSNSGDPLYPELWRACAGPLVTVPRVGDLVFYFPQGHIEQVEASMNQVAGNQMRLYDLPSKLLCRVLNVELKAETDTDEVYAQIMLMPEPEQTDVAAEKASSASAASPRPAVRSFCKTLTASDTSTHGGFSVLRRHADECLPPLDMTQSPPTQELVAKDLHGMEWRFRHIFRGQPRRHLLQSGWSVFVSSKRLVAGDAFIFLRGENGELRVGVRRAMRQLSNVPSSVISSQSMHLGVLATAWHAINTKSMFTVYYKPRTSPSEFIIPYDQYMESVKNNYSIGMRFRMRFEGEEAPEQRFTGTIVGCENLDPLWPDSSWRYLKVRWDEPSTIPRPDRVSPWKIEPASSPPVNPLPLSSRVKRPRQNAPPPSPEASVLTKESAAKIDIDSAQTQHQNSVLQGQEQMTLRNNLTESNDSDSTVQKPMMWSPSPNGKAHTNFQQRPAMDSWMPMGRRETDFKDSRSAFKDARTASQSFRDTQGFFMQAYDDNHHRLSFNNQFQDQGSAHRFADPYFYMAQQPSLTVESSTRTQTANNDLRFWGDQNSIYGNPGDQQQQGFSFGQNPSSWLNQPFPQVEQPRVVRPHATVAPFDLEKTREGSGFKIFGFQVDTTSPSPAQLSSPLCAIREHVVQTRPSAPVNELQPVQNECLPEGSVSTAGTATENEKNIQQAQPSSKDIQSKSQGASTRSCTKVV

SEQ ID NO: 6 - protein, 809 aa, Brachypodium distachyon
MAPPPQGPSAGDPLFNELWHACAGPLVTVPRVGDLVFYFPQGHIEQVEASMNQVADNQMRLYDLPSKLLCSVINVELKAEADTDEVYAQVMLIPENDQNEMAVEKSSSKAATTLAKPAVRSFCKTLTASDTSTHGGFSVLRRHADECLPPLDMTQSPPTQELVAKDLHGMDWRFRHIFRGQPRRHLLQSGWSVFVSSKRLVAGDAFIFLRGESGELRVGVRRAMRQLSNVPSSVISSHSMHLGVLATAWHAINTKSMFTVYYKPRTSPSEFIIPYDQYMESVKNNYSIGVRFRMRFEGEEAPEQRFTGTIIGSENLDPLWPESSWRSLKVRWDEPSTIPRPDRVSPWKIEPASSPPVNPLPLSRVKRPRPNVPPASPESSALTKEGATKVDVDSAQAQRNQTSMVLQGQEPMTLRSNNLTDSNDSDATVQKPMMWSPSPNIGKSRPLTFQQRPSMDNWMQLGRRETDYKDASSGAQSFGDSPGFFVQTYDEAPNHLASFKNQFQDQGPARHFSEPYFFMHQQPNLTVDSSTKMHPETNELHFWNGQNTVYGHSGDQSQGFRFEEHPSNWSSQQFSRVEQPRVIRPHASIAPVDLEKTREGSGFKIFGFKVDTASAPTNHLSSPMAATHEPALQTQPSASLNQLQHAQTDCFPEVSVSTGGTNENEKSIQQAPQSSKDVQSKSHGASTRSCTKVHKQGVALGRSVDLSKFSDYDELKAELDKMFEFDGELMSSNKNWQIVYTDNEDDMMLVGDDPWGEFCSIVRKICIYTKEEVQKMNSKLSAPRKEDGAAPCDGANENAALPAPGNLDN

SEQ ID NO: 7 - protein, 742 aa, Triticum aestivum
MNQVAGNQMRLYDLPSKLLCRVINVELKAEADTDEVYAQVMLMPEPEQNEMAVDKSTSTTGATPPRPAVRSFCKTLTASDTSTHGGFSVLRRHADECLPPLDMTQSPPTQELVAKDLHGMDWRFRHIFRGQPRRHLLQSGWSVFVSSKRLVAGDAFIFLRGESGELRVGVRRAMRQLSNVPSSVISSHSMHLGVLATAWHAINTKSMFTVYYKPRTSPSEFIIPYDQYMESVKNNYSIGMRFRMRFEGEEAPEQRFTGTIVGSENLDQLWPESNWRSLKVRWDEPSTIPRPDRVSPWKIEPASSPPVNPLPLSRVKRPRPNVPPVSPESSVLTKEGATKIDMDSAQAQQRNQNSMVLQGQEHMTLRTNNLTGSNESDATVQKPMMWSPSPNIGKNHASAFQQRPSMDNWMQLGRCDASSGAQSFGDSQGFFMQTFDEAPNRHGSFKNQFQDHSSARHFSDPYTKMQTEANEFHFWNSQSTVYGNPRDQSQGFRFEEHPSNWLRQQQLSPVEQPRVIRPHASIAPVDLEKTREGSGFKIFGFKVDTTSAPSNHLSSTTAAIHEPVLQTQASASLTQLQHAHADCIPELSVSTAGTTENEKSIQQTPHSSKDVQSKSHGASTRSCTKVHKQGVALGRSVDLSKFGDYDELTAELDRMFEFDGELMSSNKDWQIVYTDPEGDMMLVGDDPWEEFCNIVRKIFIYTKEEVQKMNSKSSTPRKEEGSADADGANEKAHLATSSHLDN

SEQ ID NO: 8 - protein, 796 aa, Triticum aestivum
MPPAAMAPPQAPSAGDPLYDELWHACAGPLVTVPRVGDLVFYFPQGHIEQVEASMNQIAGNQMRLYDLPSKLLCRVINVELKAEADTDEVYAQVMLMPEPEQNEMAVDKSTSTTGATPPRPAVRSFCKTLTASDTSTHGGFSVLRRHADECLPPLDMTQSPPTQELVAKDLHGMDWRFRHIFRGQPRRHLLQSGWSVFVSSKRLVAGDAFIFLRGESGELRGVRRAMRQLSNVPSSVISSHSMHLGVLATAWHAINTKSMFTVYYKPRTSPSEFIIPYDQYMESVKNNYSIGMRFRMRFEGEEAPEQRLTGTIVGSENLDQLWPESNWRSLKVRWDEPSTIPRPDRVSPWKIEPASSPPVNPLPLSRVKRPRPNVPPVSPESSVLTKEVAAKIDMDSAQAQQRNQNNMVLQGQEHMTLRTNNLTASNESDATVQKPMMWSPSPNIGKNHASAFQQRPSMDNWMQLGRCDASSGAQSFGDSQGFFMQTFDEAPNRHGSFKNQFQDHSSARHFSDPYTKMQTEASEFHFWNSQSTVYGNPRDQSQGFRFEEHPSNWLRQQQQFSPVEQPRVIRPHASIAPVDLEKTREGSGFKIFGFKVDTTSAPSNHLSSTMAAIHEPVLQTQASASLTQLQHAHADCIPELSVSTAGTTENEKSIQQAPHSSKDVQSKSHGASTRSCTKVHKQGVALGRSVDLSKFGDYDELTAELDRMFEFDGELMSSNKDWQIVYTDPEGDMMLVGDDPWEEFCNIVRKIFIYTKEEVQKMNSKSSTPRKEEGSADADGANEKAHPATSSHLDN

SEQ ID NO: 9 - protein, 824 aa, Triticum aestivum
MPPAAMPPPPPPPQGASTGDPLYDELWHACAGPLVTVPRVGDMVYYFPQGHIEQVEASMNQVAANQMRLYDLPSKLLCRVLNVELKAEADTDEVYAQVMLMPEPEQSETTTEKSSPATGGATPARPAVRSFCKTLTASDTSTHGGFSVLRRHADECLPPLDMTQSPPTQELVAKDLHGMEWRFRHIFRGQPRRHLLQSGWSVFVSSKRLVAGDAFIFLRGESGELRVGVRRAMRQLSNIASSVISSHSMHLGVLATAWHAINTKTMFTVYYKPRTSRSEFIIPYDKYMESVKNIYSIGTRFKMRFEGEEAPEQRFTGTIVGSDNLDQLWPESSWRSLKVRWDESSTIPRPDRVSPWEIEPASSPPVNPLPISRPKRSRPNVPPASPESSVRTKEGATKADMDCAQAQRNQNNTALPGQEQRSNKLTDINDFDASVQKPMMWSPPPPPNIGKNNPLTFQQRPSVHNSFQLGRRETDFKDASSGAQPFGDSLGFFMQTTFDEAPNRLSSFKNQFQDHSYARQYADPYLFMHQQPSLTVESSRKMHTENNEMHFWNGPSTAYSNSRDQAQDFRFTEHPSNWLSPQFSRAEQPRVIRPHASIAPIELEKTTEGSDFKIFGFKVDTASAGFNHLNSPMAGTHEPVLQTQPSVSLDHLQTDCSPEVSLSIAGTTENEKNMQQCPQSSKDVQSKSHGASTRSCTKVHKQGVALGRSVDLSKFVDYDELTAELDKMFEFDGELLSSNKNWQIVYTDNEGDMMLVGDDPWEEFCSIVRKICIYTKEEVQKMNSKPSGPKKEEGSVDGDGATEKAHLPASSDLNSSQLGCVKAD

SEQ ID NO: 10 - protein, 805 aa, Zea mays
MPPATAMAPPSQPPSNSGDPLYPELWRACAGPLVTVPRVGDLVFYFPQGHIEQVEASMNQVAGNQMRLYDLPPKLLCRVLNVELKAETDTDEVYAQIMLMPEPEQTDVPAEKPSSAPAASPRPAVRSFCKTLTASDTSTHGGFSVLRRHADECLPPLDMTQSPPTQELVAKDLHGMEWRFRHIFRGQPRRHLLQSGWSVFVSSKRLVAGDAFIFLRGENGELRVGVRRAMRQLSNVPSSVISSQSMHLGVLATAWHAINTKSMFTVYYKPRTSPSEFIIPYDQYMESVKNNYSIGMRFRMRFEGEEAPEQRFTGTIVGCENLDPLWPDSSWRYLKVRWDEPSTIPRPDRVSPWKIEPASSPPVNPLVHSSRAKRPRQNVPPPSLESSVLTKEGATKVEIDSAQTQHQNSVLQGQEQMTLRNNLTESADSDSTVQKPMMWSPSLNGKAHTHFQQRPAMDNWMPLGRRENDFKDTRSAFKDARTASQSFGDTQGFFVQAYDDNHHRLSFKNQGSTHRFADPYFYMPQQSSVTVESSTRTQTANNDLCFWGDQNAMYGNPSDQQQGFNFGQNPSSWLNQPFPQVEQPRVVRPHATVAPFDLEKTREGSGFKIFGFQVDTTSPSPVQLSSPLPAIQEHVLQTRPSAPVNELQPVQIECLPEGSVSTAGTATENIQQAPQSSKDIQSKSQGASTRSCTKVHKQGVALGRSVDLSKFTDYGELQAELDKMFDFEGELVSGSQNWQIVYTDDEGDMMLVGDDPWEEFCSIVRKIYIYTKEEVQKMNSKSAAPRKEESPAAGGGGGCAAATNE

SEQ ID NO: 11 - DNA, 1984 bp, Zea mays
gagtccttgcgactgcatggcacgccattaacacgaagtccatgttcactgtctactacaagcctaggacgagcccttcagagttcatcataccgtacgatcaatatatggagtcggtaaaaaataattattctattggcatgagattcaggatgaggtttgaaggagaagaggcgccagagcagaggtttactggtacaatagttggttgtgaaaatcttgacccactatggcctgattccagttggagatacttgaaggtgcgttgggatgagccttctactatcccgcggccagatagggtctctccttggaagatagagcctgcttcatcgcctcctgttaatccactcgtgcattcttctagagcaaaaagacctagacaaaacgttcctccaccttcacttgaatcatctgttctcacaaaagaaggtgcaaccaaggtcgaaatcgattcagctcaaacacaacaccaaaattcggtcttgcaaggtcaggagcagatgaccttgaggaacaacctgactgaaagcgctgattctgattccactgttcagaagccgatgatgtggtccccatcactcaatgggaaagcccacactcattttcagcagagacctgctatggataattggatgccattgggaaggcgtgaaaatgacttcaaggacacccgttctgccttcaaggatgcccgcaccgcctctcaatcatttggagatacacagggtttttttgtgcaagcttatgatgacaatcatcaccgtctctctttcaaaaatcagggttcaactcatcgcttcgcagatccgtacttctatatgcctcaacaatcgtcagtgacggttgaatcaagcacaaggacacaaacagcaaacaatgacttgtgtttctggggtgaccagaatgctatgtacggcaatccaagtgaccaacaacagggtttcaactttggacaaaacccatcaagttggttgaaccagccatttccccaggttgaacagccacgagtggtcaggcctcatgcaacggttgctccatttgatctggagaagactagagaaggaagtggcttcaagatttttgggttccaagttgatacaaccagtccatctcctgtccaattgagctctccattgcccgctatccaggagcatgtgttacaaactcgaccatcggcaccggtgaatgaattgcaacctgtgcaaattgagtgcttgcctgagggatctgtaagcacagctggaactgcaacggagaacattcagcaagccccgcagagttcaaaagatatccaaagcaagtcccagggtgcttcaacaaggagctgtacaaaggttcataagcaaggagtcgcacttggcaggtcggtggacctctctaaattcaccgactacggcgaactccaagcagagctggacaagatgtttgacttcgagggtgaattggtatctggcagccaaaactggcagatcgtctataccgacgacgagggcgatatgatgcttgtgggagacgacccgtgggaagagttctgcagcatagtgcgcaagatctacatctacacgaaggaggaggtccagaagatgaactcgaaatcagctgcgccaaggaaggaggaatctccggcagcgggcggcggaggaggttgcgccgccgccacaaacgagtagtatggtggtttgctcttggtggcaagttgaggccaaggttttactactgctattgttattgctgatttgctctccccgccttccggtgtaaatatctgtgtgtacccagtcccggccttgtgctagtaagtagcaagcagtgttaccttttggtattttaaccagcagctaagttcgcggcattttagtccctgtaagcggtaagcagctctgtagcgtgagatagatggccaagcgccggtacgtacgtgtagtgtctggtcttctcactgagtgtactataaagagttaatataggagctgaccctgtg

SEQ ID NO: 12 - DNA, 792 bp, Zea mays
cacatcaaaattcggtcttgcaaagtcaagagcagatgagctttaggaacaacctgactgaaagcaccgactctgattccactgttcagaagcagatgatgtggtccccatcacccaatgcgaaagttcacactaattttcagccgagacctgctatggataactggatgccattgggaaggcgtgaaactgacttcaaggacacccgttctgccttcaaggatgcccgcaccgcttctcaatcatttggagatacacaggggttctttatgcaagcttatgacgacagtcgtcaccgtctttctttcaacaatcagtttcaagatcagggttcagctcatcgctttgcagatccatacttctatatgcctcaacaaccttctctgacggttgaatcgggcacaaggacgcaaacagcgaacaatgacttgcgtttttggagtgagcggaattcgatgtatgggaatccaagtgaccaacagcagggtttcagctttgggcaaaacccatcaagttggttgaaccagccacatccccaggttgaacagtcacgagtggtcaggccgcatgcaacggttgctccatttgatttggagaagactagagaaggcagtgagttcaagatttttggtttccaagttgatacaaccaatccatcacctgtccaattgagctctccattgtctgctatccgagagcatgtggtacaaactcgaccatcggcaccagtgaatgaatcgcaacctgtgcaaatcgagtgcttgcctgaggtgtccgtaagcacagctggaa

SEQ ID NO: 13 - DNA, 215 bp, Triticum aestivum
ttccgtgggcaacctaggaggcatctccttcagagcggttggagtgtgtttgtcagttccaaaaggcttgtagctggggatgccttcatttttctcagaggagagagtggtgagcttcgtggtgttaggcgggctatgagacagctgtccaacgtgccttcttcagtcatatctagtcatagcatgcaccttggagtccttgcaactgcatggca

SEQ ID NO: 14 - DNA, 250 bp, Oryza sativa
ccgcgtctgacaccagcacgcacggcggcttctctgtactccgccgccacgctgatgagtgcctccctcccctggatatgacccagtcgccacccacacaggagctcgtggccaaggatctgcacagcatggactggcgcttccgtcacatctttcgcgggcaaccgaggaggcatctccttcagagtggttggagtgtgtttgtcagttccaaaaggcttgttgctggggatgctttcattttcctcag

SEQ ID NO: 15 - DNA, 250 bp, Sorghum bicolor
gacctcgtcttctactttccccagggacacatcgagcaggtggaggcgtccatgaaccaggtcgccgggaaccagatgcgcctctacgatctgccctccaagctgctctgccgcgtgctcaacgtcgagcttaaggcggagacggacaccgacgaggtctacgcgcagatcatgctcatgccagagcccgagcaaaccgatgtggcggctgagaaggcgagttctgcgtccgctgcgtcgccgaggccag

SEQ ID NO: 16 - DNA, 250 bp, Brachypodium distachyon
acctcgtcttctacttcccgcagggccacatcgagcaggtggaggcctccatgaaccaggtcgccgacaaccagatgcgcctctacgatctgccttccaagctgctctgcagcgtcatcaacgtcgagctcaaggcggaggccgacaccgacgaggtctacgcgcaagtcatgctcatcccggagaacgatcaaaacgagatggcggtggagaagtcgagctccaaagcagccaccacgctagcaaagcc

SEQ ID NO: 17 - DNA, 502 bp, Artificial
  CSVMV promoter
aaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaattt

SEQ ID NO: 18 - DNA, 4432 bp, Artificial
  RNAi construct for maize
tcgagaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtccctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgataagcttgatctccaccctagaactagtggatcccccgggctgcaggaattcgatatcaccactttgtacaagaaagctgggtctagatatctcgagtgcggccgcgggaattcgattcacatcaaaattcggtcttgcaaagtcaagagcagatgagctttaggaacaacctgactgaaagcaccgactctgattccactgttcagaagcagatgatgtggtccccatcacccaatgcgaaagttcacactaattttcagccgagacctgctatggataactggatgccattgggaaggcgtgaaactgacttcaaggacacccgttctgccttcaaggatgcccgcaccgcttctcaatcatttggagatacacaggggttctttatgcaagcttatgacgacagtcgtcaccgtctttctttcaacaatcagtttcaagatcagggttcagctcatcgctttgcagatccatacttctatatgcctcaacaaccttctctgacggttgaatcgggcacaaggacgcaaacagcgaacaatgacttgcgtttttggagtgagcggaattcgatgtatgggaatccaagtgaccaacagcagggtttcagctttgggcaaaacccatcaagttggttgaaccagccacatccccaggttgaacagtcacgagtggtcaggccgcatgcaacggttgctccatttgatttggagaagactagagaaggcagtgagttcaagatttttggtttccaagttgatacaaccaatccatcacctgtccaattgagctctccattgtctgctatccgagagcatgtggtacaaactcgaccatcggcaccagtgaatgaatcgcaacctgtgcaaatcgagtgcttgcctgaggtgtccgtaagcacagctggaaaatcactagggttcctttaaagcctgcttttttgtacaaacttgtgatatcaagcttcgtggatccagatggccgcggcatccaggtaaaacaaacaaaaacggatctgatgcttccattcctccgtttctcgtagtagcgcgcttcgatctgtgggtggatctgggtgatcctggggtgtggttcgttctgtttgatagatctgtcggtggatctggccttctgtggttgtcgatgtccggatctgcgttttgatcagtggtagttcgtggatctggcgaaatgttttggatctggcagtgagacgctaagaatcgggaaatgatgcaatattaggggggtttcggatggggatccactgaattagtctgtctccctgctgataatctgttcctttttggtagatctggttagtgtatgtttgtttcggatagatctgatcaatgcttgtttgttttttcaaattttctacctagggtgtataggaatggcatgcggatctggttggattgccatgatccgtgctgaaatgcccctttggttgatggatcttgatattttactgctgttcacctagatttgtactcccgtttatacttaatttgttgcttattatgaatagatctgtaacttaggcacatgtatggacggagtatgtggatctgtagtatgtacattgctgcgagctaagaactatttcagagcaagcacagaaaaaaatatttagacagattgggcaactatttgatggtctttggtatcatgctttgtagtgctcgtttctgcgtagtaatcttttgatctgatctgaagataggtgctattatattcttaaaggtcattagaacgctatctgaaaggctgtattatgtggattggttcacctgtgactccctgttcgtcttgtcttgataaatcctgtgataaaaaaaattcttaaggcgtaatttgttgaaatcttgttttgtcctatgcagcctgatccatcctgcaggaattcctcgatcgaggtcgacggtatcgataagcttgatatcacaagtttgtacaaaaaagcaggctttaaaggaaccctagtgattttccagctgtgcttacggacacctcaggcaagcactcgatttgcacaggttgcgattcattcactggtgccgatggtcgagtttgtaccacatgctctcggatagcagacaatggagagctcaattggacaggtgatggattggttgtatcaacttggaaaccaaaaatcttgaactcactgccttctctagtcttctccaaatcaaatggagcaaccgttgcatgcggcctgaccactcgtgactgttcaacctggggatgtggctggttcaaccaacttgatgggttttgcccaaagctgaaaccctgctgttggtcacttggattcccatacatcgaattccgctcactccaaaaacgcaagtcattgttcgctgtttgcgtccttgtgcccgattcaaccgtcagagaaggttgttgaggcatatagaagtatggatctgcaaagcgatgagctgaaccctgatcttgaaactgattgttgaaagaaagacggtgacgactgtcgtcataagcttgcataaagaacccctgtgtatctccaaatgattgagaagcggtgcgggcatccttgaaggcagaacgggtgtccttgaagtcagtttcacgccttcccaatggcatccagttatccatagcaggtctcggctgaaaattagtgtgaactttcgcattgggtgatggggaccacatcatctgcttctgaacagtggaatcagagtcggtgctttcagtcaggttgttcctaaagctcatctgctcttgactttgcaagaccgaattttgatgtgaatcgaattcccgcggccgcactcgagatatctagacccagctttcttgtacaaagtggtgatatcgaattcctgcagcccgggggatccactagttctagagcggccgccaccgcggtggagatcctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg

SEQ ID NO: 19 - DNA, 24 bp, Artificial
  primer
cacatcaaaattcggtcttgcaaa

SEQ ID NO: 20 - DNA, 21 bp, Artificial
  primer
ttccagctgtgcttacggaca

SEQ ID NO: 21 - DNA, 20 bp, Artificial
  primer
agccttctactatcccgcgg

SEQ ID NO: 22 - DNA, 24 bp, Artificial
  primer
aggctctatcttccagggagagac

SEQ ID NO: 23 - DNA, 22 bp, Artificial
  primer
tggtgaagatgataccgaccaa

SEQ ID NO: 24 - DNA, 20 bp, Artificial
  primer
agcaaccgtctgcctcatgt

SEQ ID NO: 25 - DNA, 20 bp, Artificial
  primer
tgctggacctcgattggttt

SEQ ID NO: 26 - DNA, 19 bp, Artificial
  primer
gtcctgcatcaaccgcttg

SEQ ID NO: 27 - protein, 826 aa, Hordeum vulgare
MPPAAMPPPPPPPQGTSTGDPLYDELWHACAGPLVTVPRVGDMVYYFPQGHIEQVEASMNQVAANQMRLYDLPSKLLCRVLNVELKAEADTDEVYAQVMLMPEPEQSEAAATTTEKSSSATGGTMPARPAVRSFCKTLTASDTSTHGGFSVLRRHADECLPPLDMTQSPPTQELVAKDLHGMEWRFRHIFRGQPRRHLLQSGWSVFVSSKRLVAGDAFIFLRGESGELRVGVRRAMRQLSNIASSVISSHSMHLGVLATAWHAINTKTMFTVYYKPRTSRSEFIIPYDKYTESVKNIYSIGTRFKMRFEGEEAPEQRFTGTIVGSDNLDQLWPESSWRSLKVRWDESSTIPRPDRVSPWEIEPASSPPVNPLPLSRAKRSRPNVPPASPESSVRTKEGATKADMDCAQAQRNQNNTVLPGHEQRSNKLTDINDFDATVQKPMVWSTPPPNIGKNNPLTFQQRPSVHNSIQLRRREADFKDASSGAQHFGDSLGFFMQTTFDEAPNRLGSFKNQFQDHSYARQFADPYLFMHQQPSLTVESSRKMHTENNELHFWNGPGTVYGNSIDHVQDFRFKEHPSNWLSPQFSRAEQPRVIRPHASIAPIELEKTTEGSDFKIFGFKVDTASAGFNHLNSPMAATHEPVLQTQPSVSLDHLQTDCSPEVSLSIAGTTDNEKNIQQCPQSSKDVQSKSHGASTRSCTKVHKQGVALGRSVDLSKFVDYDELTAELDKMFDFDGELMSSNKNWQIVYTDNEGDMMLVGDDPWEEFCSMVRKICIYTKEEVQKMNSKPSDARKEEGSVEGDGATEKVHLPVSSDLNSSQLGCVKAD

SEQ ID NO: 28 - protein, 859 aa, Arabidopsis thaliana
MASSEVSMKGNRGGDNFSSSGFSDPKETRNVSVAGEGQKSNSTRSAAAERALDPEAALYRELWHACAGPLVTVPRQDDRVFYFPQGHIEQVEASTNQAAEQQMPLYDLPSKLLCRVINVDLKAEADTDEVYAQITLLPEANQDENAIEKEAPLPPPPRFQVHSFCKTLTASDTSTHGGFSVLRRHADECLPPLDMSRQPPTQELVAKDLHANEWRFRHIFRGQPRRHLLQSGWSVFVSSKRLVAGDAFIFLRGENGELRVGVRRAMRQQGNVPSSVISSHSMHLGVLATAWHAISTGTMFTVYYKPRTSPSEFIVPFDQYMESVKNNYSIGMRFKMRFEGEEAPEQRFTGTIVGIEESDPTRWPKSKWRSLKVRWDETSSIPRPDRVSPWKVEPALAPPALSPVPMPRPKRPRSNIAPSSPDSSMLTREGTTKANMDPLPASGLSRVLQGQEYSTLRTKHTESVECDAPENSVVWQSSADDDKVDVVSGSRRYGSENWMSSARHEPTYTDLLSGFGTNIDPSHGQRIPFYDHSSSPSMPAKRILSDSEGKFDYLANQWQMIHSGLSLKLHESPKVPAATDASLQGRCNVKYSEYPVLNGLSTENAGGNWPIRPRALNYYEEVVNAQAQAQAREQVTKQPFTIQEETAKSREGNCRLFGIPLTNNMNGTDSTMSQRNNLNDAAGLTQIASPKVQDLSDQSKGSKSTNDHREQGRPFQTNNPHPKDAQTKTNSSRSCTKVHKQGIALGRSVDLSKFQNYEELVAELDRLFEFNGELMAPKKDWLIVYTDEENDMMLVGDDPWQEFCCMVRKIFIYTKEEVRKMNPGTLSCRSEEEAVVGEGSDAKDAKSASNPSLSSAGNS

(19) United States
(12) Patent Application Publication
(10) Pub. No.: US 2015/0007360 A1
(43) Pub. Date: Jan. 1, 2015

(54) METHOD FOR PLANT IMPROVEMENT

(71) Applicants: Biogemma, Paris (FR); Genoplante-Valor, Paris (FR)

(72) Inventors: Wyatt Paul, Chappes (FR); Jacques Rouster, Chappes (FR)

(73) Assignees: Biogemma, Paris (FR); Genoplante-Valor, Paris (FR)

(21) Appl. No.: 14/365,471

(22) PCT Filed: Dec. 14, 2012
(86) PCT No.: PCT/EP2012/075523

§ 371 Date: Jun. 13, 2014

(30) Foreign Application Priority Data
Dec. 14, 2011 (EP) 11306664.1

(51) Int. Cl. C12N 15/82; C12Q 1/68; C12N 15/01

(52) U.S. Cl. CPC C12N 15/8218; C12N 15/8261; C12N 15/01; C12Q 1/6895; C12Q 2600/13; C12Q 2600/158
USPC 800/276; 800/320; 800/320.1; 800/290; 800/286; 435/6.12

(57) ABSTRACT

The invention relates to the field of plant improvement, in particular of the improvement of yield for cereal plants, by down-regulation of the gene coding for Arf2.

The invention relates to the field of plant improvement, in particular of the improvement of yield for cereal plants.

Cereals are grasses (members of the monocot family Poaceae, also known as Gramineae) cultivated for the edible components of their grain, composed of the endosperm, germ and bran.

In the context of the present invention a cereal shall mean in particular maize, rice, wheat, barley, sorghum, millet, oats, rye, triticale (hybrid of wheat and rye), fonio, as well as two pseudocereals, namely buckwheat and quinoa.

In agriculture, yield is the amount of product harvested from a given acreage (eg weight of seeds per unit area). It is often expressed in metric quintals (1 q=100 kg) per hectare in the case of cereals. It is becoming increasingly important to improve the yield of seed crops to feed an expanding population and, more recently, for biofuel production. One strategy to increase the yield is to increase the seed size, provided that there is not a concomitant decrease in seed number.

Some authors have described plants which present larger grains than control plants.

In particular, WO 2005/085453 deals, in its introduction, with the problem of increasing yield through increase of the seed size.

This patent application reviews different strategies in order to increase seed size, and cites some patent applications related to genes that are supposed to be of interest for increasing seed size. This patent application describes a mutant obtained in Arabidopsis thaliana, which presents larger seeds than controls.

This application speculates on the possibility to inhibit the gene mnt (as described), which is actually the arf2 gene (for auxin response factor 2) in various plants in order to increase seed size.

Nevertheless, it is to be noted that this application does not provide experimental data on plants others than A. thaliana (some incomplete data is provided for Brassica napus) and that it specifies that the mature seeds of monocots and in particular of cereals, have a different structure than the Arabidopsis seeds (although there may be some similar steps during the seed development between rice and Arabidopsis).

Furthermore, the application only describes the Arabidopsis and B. napus sequences and only provides information on a rice partial DNA sequence.

The examples describe that the mnt mutant plants are self-sterile late in development, due to floral abnormalities (page 25, lines 1-2). The examples also indicate that the RNAi constructs made in Arabidopsis lead to plants that present the same phenotype than the mutants (page 39, paragraph 4c). It is also interesting to note that overexpression of mnt gene in Arabidopsis leads to plants presenting the same phenotype as the mutant (closed flowers, larger seeds), whereas there is no diminution of the level of MNT expression (page 45).

Finally, example 12c (pages 56 and foll.) indicates that another gene may be more suited than mnt for producing fertile plants with larger seeds. Indeed, using the IPT1 gene allows obtaining plants with normal vegetative development which produced large seeds.

The inventor of WO 2005/085453 is also the corresponding author of Hughes et al (Plant Biotechnol J. 2008 October; 6(8):758-69. Epub 2008 Jul. 8). This article further describes the mnt mutants of WO 2005/085453. The mnt gene is now correctly named arf2.

This article confirms that the arf2 mutants have some kind of infertility. It also indicates that neither heterozygous nor homozygous mutants show any increase in yield. These heterozygous or homozygous mutants actually show a decrease in yield. In order to correct infertility problems, the authors engineered plants with a targeted expression of the arf2 gene in sepals and petals to restore the opening of the flowers and the number of seeds.

The authors observed that homozygous plants with the restoration of function in these organs have larger seeds than wild. However, the yield is lower (FIG. 3c) due to the production of fewer seeds per plant. (page 763, right column). The authors calculated the Harvest Index, which is also reduced.

In conclusion, this article indicates that the arf2 gene mutants have larger seeds, but that infertility is observed in the homozygous mutants (because of problems of flowering), and that yield is decreased for all tested plants (homozygous mutants, heterozygous mutants or homozygous mutants with partial restoration of the gene in the flowers to increase fertility).

Okushima et al (Plant J. 2005 July; 43(1):29-46) describe three arf2 mutants, with a number of phenotypic characteristics such as delay in flowering, flowers abnormal sterility as is reported in WO 2005/085453.

Analysis of the presence of the polypeptide shows that there is no protein production for the arf2-8 mutant arf2, while there is accumulation of truncated polypeptide for arf2-6 and arf2-7 mutants (FIG. 5.e).

The authors mention that the seeds of the mutants are larger than wild plants (page 35, FIG. 3.i). However, no conclusion is given on performance.

The authors also produced RNAi and overexpressing transgenic plants: they indicate that overexpression results in co-suppression, and neither the RNAi nor overexpressors (co-deleted) produce polypeptides. The phenotypes are similar to those of mutants. The results obtained on the overexpressor plants are similar to the ones reported in WO 2005/085453 with regards to the phenotype of the plants, but opposite with regards to the expression of the ARF2 protein.

Lim et al (J Exp Bot. 2010 March; 61(5): 1419-1430) performed EMS and identified the arf2 gene as being mutated in plants which have delayed senescence of leaves. These plants also have larger aerial organs, inhibition of the opening of buds, delayed flowering, and delayed senescence of roots. The authors provide no information on seed size or yield.

Article Schruff et al (Development. 2006 January; 133(2):251-61. Epub 2005 Dec. 8.) also comes from the inventor of WO 2005/085453. This article describes the mnt Arabidopsis mutant which has seeds with larger size weight. As already described this mutant also present other phenotypes characteristics such as problem for opening buds and self-pollinating.

Vert et al (Proc Natl Acad Sci USA. 2008 Jul. 15; 105(28):9829-34. Epub 2008 Jul. 3.) indicate that arf2 mutants show a number of growth defects.

Unexpectedly, the inventors have demonstrated that it is possible to increase yield in cereals, by inhibiting (down-regulating) the arf2 gene in these plants, without observing the sterility defects reported in the prior art.

In the context of the present invention, the arf2 gene is the gene that codes for an Arf2 protein (which is an orthologue of Arabidopsis thaliana protein depicted in SEQ ID NO 28), wherein alleles of said Arf2 protein are depicted by SEQ ID NO 1 (for maize), SEQ ID NO 3 (for rice), SEQ ID NO 4 or SEQ ID NO 27 (for barley), SEQ ID NO 5 (for sorghum), SEQ ID NO 6 (for Brachypodium) and SEQ ID NO 7 to SEQ ID NO 9 (which may correspond to the Arf2 proteins for the three genomes of wheat).

It is known that the sequence of the arf2 gene or of the Arf2 protein may vary within one species as there are allelic variations of the sequences for the same gene between different individuals in the same species.

Consequently, the Arf2 protein also encompasses proteins having a sequence presenting at least 95% identity, more preferably at least 98% identity, most preferably at least 99% identity with SEQ ID NO 1, SEQ ID NO 3, SEQ ID NO 4, SEQ ID NO 27, SEQ ID NO 5, SEQ ID NO 6, SEQ ID NO 7, SEQ ID NO 8 or SEQ ID NO 9.

The degree of identity is defined by comparison with the entire sequence of reference. This percentage of identity is preferably obtained by the method of Needleman and Wunsch (J. Mol. Biol. 48: 443 (1970)).

“Percentage of sequence identity” can be determined by comparing two optimally aligned sequences over a comparison window, where the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A preferred method uses the algorithm of Needleman and Wunsch.

In maize, said Arf2 protein is coded by the arf2 gene, the cDNA sequence of which is represented by SEQ ID NO 2.

cDNA sequences coding for the other Arf2 proteins depicted in SEQ ID NO 3 to SEQ ID NO 9 and SEQ ID NO 27 can easily be found by a person skilled in the art on public databases, using the tblastn software.

In a first aspect, the invention relates to a cereal comprising at least one cell which presents total or partial inhibition of the expression of a gene coding for the Arf2 protein as specified above. Said inhibition is due to the presence of a “determinant” (as described below).

As foreseen in the present invention, a total inhibition of a gene coding for the Arf2 protein in a cell indicates either that:
(i) No arf2 mRNA is detected in said cell after RNA isolation and reverse transcription or Northern Blot. In particular, total inhibition is obtained when no mRNA is detected after RNA isolation and reverse transcription according to the method described in the examples (which illustrates the method for maize).
(ii) No functional protein is produced in said cell. No functional protein is produced in absence of arf2 mRNA (see (i)). In other cases, mRNA may be present but leads to the production of a truncated protein (as an illustration when the arf2 mRNA is incomplete, in particular in case of the presence of a mutation within the gene, in an intron or in an exon). Truncated proteins can be detected by isolation of the proteins, Western Blot and detection of the size of the protein with an antibody (polyclonal or monoclonal) directed against the Arf2 protein. Okushima et al. (op. cit.) describe such strategy and the means to obtain such data. Presence of mutations in the active site of the protein (binding site of the protein to DNA, as described in Vert et al (op.cit.)) may also lead to production of a non functional protein. Wang et al (Mol Biol Rep, 2011, Jun. 11. [Epub ahead of print]) also describe precisely the structure of the ARF proteins, and the localization of the DNA binding domain.

As foreseen in the present invention, a partial inhibition of a gene coding for the Arf2 protein in a cell indicates arf2 mRNA is detected in said cell after RNA isolation and reverse transcription or Northern Blot, but at a lower level than that detected in a cell which do not bear the determinant leading to arf2 inhibition. In particular, partial inhibition is obtained when a lower level of mRNA is detected after RNA isolation and reverse transcription according to the method described in the examples (which illustrates the method for maize). In particular, partial inhibition is obtained when the level of arf2 mRNA is lower than 0.9 times, more preferably lower than 0.75 times, and more preferably lower than 0.66 times of the level of arf2 mRNA in a cell which does not bear the determinant leading to arf2 inhibition (the term “determinant” is described below). Preferably, said cell which does not bear the determinant leading to arf2 inhibition is from a plant that is isogenic to the plant from which originates the cell in which partial inhibition is to be detected. Preferably, the cells are from the same plant tissue and mRNA is isolated at the same level of development. It is indeed most preferred that the level of inhibition is compared from comparable cells, only differing from the presence or absence of the determinant.

The level of arf2 mRNA can be measured as an absolute level. It is nevertheless preferred that the level of arf2 mRNA is measured as a relative level, compared to other control genes. In this case the method to be used to measure the level of mRNA and to detect inhibition is as follows:
mRNA is isolated from tissues in which it is supposed to be inhibited and control tissues (such as leaves as an example)
Reverse transcription and real-time quantitative PCR are performed on said mRNA using primers that amplify the arf2 gene or primers that amplify control genes. These control genes are genes which are known to be usable as control in Northern Blot analysis, as their quantity level rarely varies. One can cite actin, ubiquitin 2, EF1α genes. It is preferred that at least two control genes are used, and in particular ubiquitin 2 and EF1α.
The Cp is then calculated for each amplified sample according to methods known in the art for real-time qPCR. In particular, machine used to perform real-time qPCR usually present a software which can automatically calculate this value, by calculation of the second derivative maximum.
One then calculates the value equal to 2exp (—(Cp for arf2 mRNA—Cp for control gene (or mean of Cp of the control genes). This value gives a relative level of expression for arf2 mRNA as compared to the level of expression of the control gene. If this value is higher than 1, this means that there is more arf2 mRNA than the control gene. If this value is lower than 1, this means that there is less arf2 mRNA than the control gene.
One then compares the values obtained for the cells in which arf2 is inhibited (i.e. where the determinant is present) and for the cells in which arf2 is at the basal level (i.e. where the determinant is absent). Ratio between these two values allows the determination of the level of inhibition of the arf2 gene.

In a preferred embodiment, said cereal is maize (also named corn in this document). In this case, the sequence of one allele of an Arf2 protein is represented by SEQ ID NO 1. It is to be noted that this protein is also named Arf29 in maize (Wang, 2011 op. cit.).

In a specific embodiment, said cereal also presents inhibition of the expression of the arf13 gene. In maize, the sequence of one allele of an Arf13 (according to the nomenclature of Wang) protein is represented by SEQ ID NO 10. The cDNA is represented by SEQ ID NO 11. It is clear that SEQ ID NO 10 represents the sequence of an allele of the Arf13 protein, and that the invention also covers inhibition of other alleles of the arf13 gene, which code for proteins having a sequence presenting at least 95% identity, more preferably at least 98% identity, most preferably at least 99% identity with SEQ ID NO 10.

These two maize sequences (ZmARF29 and ZmARF13, numbered according to the nomenclature of Wang) are putative Arabidopsis thaliana ARF2 protein orthologues.

Due to the similitude between Arf29 and Arf13 in maize, inhibition of both genes may occur depending whether the RNAi construct possesses long enough regions capable of hybridizing with both genes.

In a specific embodiment, said arf2 gene is inhibited in multiple cells of said cereal, wherein said inhibition in multiple cells results in an inhibition in one or multiple tissues of said cereal. In this embodiment, it is possible that the arf2 gene is not inhibited in other tissues of said cereal.

In another embodiment, said arf2 gene is inhibited in all cells of said cereal. In this case, the invention encompasses a cereal which presents an inhibition of the expression of the arf2 gene.

It is to be noted that the invention envisages that the inhibition of the arf2 gene is obtained by various methods, such as mutation of the gene or transformation of the plant with a vector that will eventually cause the inhibition.

As indicated above, the inhibition is obtained by the introduction of a “determinant” in cells of said cereal. As foreseen herein, a “determinant” causes the inhibition of the arf2 gene, is inheritable from generation to generation and is transmissible to other plants through crosses. Determinants will be described in more details below and include mutations and transgenes (introduced foreign DNA within the genome of the cells of the plant).

As indicated above, the inhibition may be said to be “total” or “full” (i.e. there is no more production of functional Arf2 protein) or “partial” (i.e. there is a decrease in the production of functional Arf2 protein, as compared with the production in a plant that does not contain the mutation or the vector causing the inhibition). It is also to be noted that inhibition does not preclude production of non-functional Arf2 protein (such as a truncated protein, in particular in case of a non-sense mutation present in the arf2 gene).

It is also possible that the cereal presents a total inhibition of the arf2 gene in some tissues, whereas there is no or only a partial inhibition in other tissues.

In an embodiment inhibition of the Arf2 expression is obtained by a mutation of the arf2 gene through insertion of a transposable element or of a T-DNA or following physical mutagenesis.

In this embodiment, expression and/or activity of ARF2 is inhibited by mutagenesis of the gene coding for said protein.

The mutagenesis of the gene can take place at the level of the coding sequence or of the regulatory sequences for expression, in particular of the promoter. It is, for example, possible to delete all or part of said gene and/or to insert an exogenous sequence.

By way of example, mention will be made of insertional mutagenesis: a large number of individuals derived from a cereal that is active in terms of the transposition of a transposable element (such as the AC or Mutator elements in maize) are produced, and the cereals in which there has been an insertion in the arf2 gene are selected, for example by PCR.

It is also possible to introduce one or more point mutations with physical agents (for example radiations) or chemical agents, such as EMS or sodium azide treatment of seed, site-directed DNA nucleases or gamma irradiation. The consequences of these mutations may be to shift the reading frame and/or to introduce a stop codon into the sequence and/or to modify the level of transcription and/or of translation of the gene. In this context, use may in particular be made of techniques of the “TILLING” type (Targeting Induced Local Lesions IN Genomes; McCALLUM et al., Plant Physiol., 123, 439-442, 2000). Such mutated cereals are then screened, in particular by PCR, using primers located in the target gene. One can also use other screening methods, such as Southern Blots or the AIMS method that is described in WO 99/27085 (this method makes it possible to screen for insertion), by using probes that are specific of the target genes, or through methods detecting point mutations or small insertions/deletions by the use of specific endonucleases (such as Cel I, Endo I, which are described in WO 2006/010646).

In this embodiment, the determinant as mentioned above is the mutation. It is indeed inheritable and transmissible by crosses.

In another embodiment, inhibition of the Arf2 expression is due to the presence in cells of said cereal of an antisense, or overexpression (leading to co-suppression), or RNAi construct. The DNA constructs used in these methods are introduced in the genome of said cereal through methods known in the art.

The transformation of vegetable cells can be achieved by any one of the techniques known to one skilled in the art.

In particular, it is possible to cite methods of direct transfer of genes such as direct micro-injection into plant embryos, vacuum infiltration or electroporation, direct precipitation by means of PEG or the bombardment by gun of particules covered with the plasmidic DNA of interest.

It is preferred to transform the cereal with a bacterial strain, in particular Agrobacterium, in particular Agrobacterium tumefaciens. In particular, it is possible to use the method described by Ishida et al. (Nature Biotechnology, 14, 745-750, 1996) for the transformation of Monocotyledons.

In particular, inhibition may be obtained by transforming the cereal with a vector containing a sense or antisense construct. These two methods (co-suppression and antisense method) are well known in the art to permit inhibition of the target gene. It is to be noted that the data obtained in Arabidopsis demonstrates that the co-suppression approach seems adapted for this gene.

One can also use the RNA interference (RNAi) method, which is particularly efficient for extinction of genes in plants (Helliwell and Waterhouse, 2003). This method is well known by the person skilled in the art, and comprises transformation of the cereal with a construct producing, after transcription, a double-stranded duplex RNA, one of the strand of which being complementary of the mRNA of the target gene.

The invention thus relates to a cereal, which is a cereal transformed with a transgene that comprises DNA construct comprising a polynucleotide chosen among:
a) a polynucleotide coding for the arf2 gene as specified above;
b) a polynucleotide which has a sequence complementary of the sequence of the polynucleotide of a);
c) a fragment of at least 12 consecutive nucleotides of a polynucleotide of a) or b), more preferably at least 20, even more preferably at least 50, the most preferably at least 100 consecutive nucleotides of a polynucleotide of a) or b), or capable of selective hybridization with a polynucleotide of a) or b);
d) a polynucleotide containing (i) a polynucleotide X comprising at least 12 nucleotides, preferably at least 15, advantageously at least 20, and even more preferably at least 50, most preferably at least 100 nucleotides capable of selective hybridization with the polynucleotide of a), and (ii) a polynucleotide Y that is complementary of said polynucleotide X,

under transcriptional control of an appropriate promoter.

Polynucleotides a) and some polynucleotides c) are suitable for inhibition by co-suppression. Polynucleotides b) and some polynucleotides c) are suitable for inhibition by antisense. Polynucleotides d) are suitable for inhibition by RNAi.

Said DNA constructs preferably comprise a nucleotide chosen from SEQ ID NO 12, SEQ ID NO 13, SEQ ID NO 14, SEQ ID NO 15 and SEQ ID NO 16. These nucleotides are in particular used for generating vectors suitable for RNAi in maize, wheat, rice, sorghum and brachypodium, respectively.

In a preferred embodiment, the cereal transformed with said DNA construct may contain said polynucleotide within its genome. In another embodiment, the transgene may have been introduced such as being expressed transiently, or through a genetic construct not integrated in the genome. Thus, agro-infiltration or any other methods, such as injection or spray, are contemplated for transient expression.

In this embodiment, the determinant as mentioned above is the DNA construct. It is to be noted that it is not necessarily present at the same locus than the arf2 gene.

It is foreseen that said nucleic acids which are in the constructs are transcribed. They are thus under the control of an appropriate promoter. One can use various promoters, among which a constitutive, tissue specific, developmentally regulated, inducible promoter.

In a preferred embodiment, said construct is under the control of a constitutive promoter. In a most preferred embodiment, said construct is a RNAi construct, under the control of a constitutive promoter.

Other suitable promoter could be used. It could be a tissue-specific promoter such as a root-specific promoter, a leaf-specific promoter, a seed-specific, a BETL specific promoter and the like. Numerous tissue-specific promoters are described in the literature and any one of them can be used.

Examples of constitutive promoters useful for expression include the 35S promoter or the 19S promoter (Kay et al., 1987, Science, 236:1299-1302), the rice actin promoter (McElroy et al., 1990, Plant Cell, 2:163-171), the pCRV promoter (Depigny-This et al., 1992, Plant Molecular Biology, 20:467-479), the CsVMV promoter (Verdaguer et al., 1998, Plant Mol Biol. 6:1129-39), the ubiquitin 1 promoter of maize (Christensen et al., 1996, Transgenic. Res., 5:213), the regulatory sequences of the T-DNA of Agrobacterium tumefaciens, including mannopine synthase, nopaline synthase, octopine synthase.

One can also cite the promoters regulated during seed development such as the HMWG promoter (High Molecular Weight Glutenin) of wheat (Anderson O. D. et al., 1989, Theor Appl Genet, 77: 689-700; Roberts et al., 1989, Plant cell, 1:569-578), the waxy, zein or bronze promoters of maize.

Promoters may come from the same species or from another species (heterologous promoters). Although some promoters may have the same pattern of regulation when there are used in different species, it is often preferable to use monocotyledonous promoters in monocotyledons and dicotyledonous promoters in dicotyledonous plants. Consequently, the promoter is preferably a promoter that comes from a monocotyledonous plant, and more preferably from a cereal.

In a preferred embodiment, the promoter is the pCSVMV promoter which is represented by SEQ ID NO 17. It is within the scope of the invention to use a sequence that may vary from SEQ ID NO 17, for the same purpose (obtaining constitutive expression of a construct). Indeed, it is possible to add, delete or change some bases of SEQ ID NO 17, without modifying the pattern or level of expression of the promoter.

In preferred embodiments said construct comprises a RNAi construct, the sequence of which is depicted by SEQ ID NO 18. One can replace the specific maize sequence in SEQ ID NO 18 (sense from 1132 to 1881, and antisense from 3156 to 3897) by any of the sequences SEQ ID NO 13, SEQ ID NO 14, SEQ ID NO 15 or SEQ ID NO 16 in order to obtain a RNAi construct usable in wheat, rice, sorghum and Brachypodium respectively.

In SEQ ID NO 18,
nucleotides 6 to 507 represent the CSVMV promoter
nucleotides 411 to 417 represent the TATA box
nucleotide 445 represent the beginning of the Open reading Frame
nucleotides 544 to 1105 represent the rice actin intron
nucleotides 1132 to 1923 represent the first part of the RNAi construct
nucleotides 2009 to 2900 represent the rice tubulin intron
nucleotides 3156 to 3947 represent the second part of the RNAi construct
nucleotides 4024 to 4532 represent the AtSac66 terminator.

It is clear that the cereals used in the methods described below are cereals according to the invention, in which the gene arf2 (or the production of the Arf2 protein) is inhibited, either totally or partially, in all cells or in specific tissues, totally in some tissues whereas not at all or only partially in other tissues, or with the same level of inhibition in all tissues.

These cereals are obtained as described above. They can easily be characterized using methods known in the art. One can cite RNA extraction and Northern Blot which makes it possible to determine an inhibition in the level of arf2 mRNA. RNA extraction may be performed tissue by tissue and/or at various stages of development of the cereal, which makes it possible to determine the pattern of inhibition in particular when RNAi is used, with promoter having a specific pattern of expression (whether localized in specific tissues, or at different stages of development).

Alternatively, one can extract the whole protein content and perform a Western Blot in order to reveal the presence, absence or quantity of Arf2 protein. As usual, the quantity of Arf2 protein is preferably determined by comparison with the quantity of Arf2 protein in the same tissue of an isogenic plant which does not contain the determinant causing arf2 inhibition.

In the most preferred embodiment, the cereal according to the invention are such that the arf2 gene is partially inhibited, due to the presence of a transgene (DNA construct) within the cells of said cereal, which induces partial inhibition through antisense, co-suppression or RNAi methods, and wherein said transgene is under the control of a constitutive promoter.

The invention also relates to various methods of using the cereals of the invention.

In particular, the invention encompasses a method for improving yield in a cereal, comprising inhibiting the expression of a gene coding for the Arf2 protein as specified above in said cereal. In this method, the cereal with the inhibited Arf2 gene presents a higher yield than a second cereal not having an inhibited Arf2 gene, after sowing and harvest. In this method, it is clear that the increase of yield is checked by sowing and harvesting of a multiplicity of cereals that present inhibition of the arf2 gene, the yield of which is then compared with the yield obtained with a second group of cereals not presenting said inhibition, and this under the same culture conditions (sowing and harvest at the same time, on comparable parcels, use of the same amount of fertilizers, water . . . ). It is also clear that comparison is to be performed on a second group of cereals that is isogenic to the cereals having the inhibited arf2 gene. This “isogenic” cereal differs from the cereal having an inhibited arf2 gene at very few loci (less than 20, more preferably less than 10), and does not carry the determinant leading to inhibition of the arf2 gene (said determinant being the mutation in the arf2 gene (coding DNA or regulatory sequences) or the construct leading to inhibition of expression of the gene or protein). This cereal can also be called “virtually isogenic”.

The invention also relates to a method for producing a cereal that can be used in a selection process for obtaining a cereal with improved yield, comprising the step of inhibiting the expression of a gene coding for the Arf2 protein in said cereal. The inhibition of the arf2 gene can be performed by any method as described above.

Indeed, it is often preferable to sow and harvest cereals lines that have been optimized, in particular for the location in which they are cultured. Consequently, one will perform the genetic modifications (mutations, introduction of foreign DNA) in order to obtain a material that is then used for selection process. The eventual lines are obtained by introgressing the determinant leading to inhibition of the arf2 gene in reference lines having otherwise agronomic quality characteristics.

The introgression of the characteristic is in particular carried out by selection, according to methods known in the art (crossing and self-pollination). The plants are in particular selected using molecular markers.

The principle is recalled below:

A series of back crosses are performed between the elite line (in which one wishes to introduce the determinant) and a line that already carries said determinant (the donor line). During the back crosses, one can select individuals carrying the determinant and having recombined the smallest fragment from the donor line around the determinant. Specifically, by virtue of molecular markers, the individuals having, for the markers closest to the determinant, the genotype of the elite line are selected.

In addition, it is also possible to accelerate the return to the elite parent by virtue of the molecular markers distributed over the entire genome. At each back cross, the individuals having the most fragments derived from the recurrent elite parent will be chosen.

The invention thus also encompasses a method for selecting a cereal comprising the step of

a) selecting, in a population of cereals, the cereals in which the arf2 gene is inhibited.

Said selected cereal is suitable to be used, and can later be used in a selection process for obtaining a cereal with improved yield.

In a specific embodiment, said population of cereal is the progeny obtained from a cross between a first cereal line in which the arf2 gene is inhibited and a second cereal line in which the arf2 gene is not inhibited.

Said inhibition of the arf2 gene is caused by the presence of a determinant (mutation or foreign DNA as described above) within the genome of the cereal. Said selection in step a) is thus performed by identification, in the genome of said cereal, of the presence of said determinant causing arf2 inhibition (either directly through analysis of the genomic DNA of the cereal, or indirectly through analysis of the presence or absence of the products that should be obtained from the arf2 gene (mRNA from the arf2 gene and/or presence or absence of a functional or truncated Arf2 protein)).

In a specific embodiment, the selection in step a) is performed through the use of a marker that is specific to the determinant leading to the inhibition of said arf2 gene. In this embodiment, step a) is thus preferably preceded by a step comprising genotyping said population of cereals.

In a specific embodiment, the selection in step a) is preceded by a step comprising extracting the RNA from the individuals in said population and performing a Northern Blot (or an equivalent method) in order to identify the cereals in which the production of mRNA from the arf2 gene is inhibited. RNA may be extracted from specific tissues only.

In a specific embodiment, the selection in step a) is preceded by a step comprising extracting proteins from the individuals in said population and performing a Western Blot (or an equivalent method) in order to identify the cereals in which the production of Arf2 protein is inhibited. Protein may be extracted from some specific tissues only.

The invention also comprises a method for obtaining a cereal exhibiting increased yield, comprising the step of introgressing the determinant responsible for the inhibition of the arf2 gene into said cereal.

This method comprises the steps consisting in
a) crossing a first cereal line that has said determinant with a second cereal that does not have said determinant,
b) selecting, in the progeny obtained, the cereals in which the arf2 gene is inhibited and which present the best genome ratio as regards said second cereal
c) performing a back cross of said selected cereals with said second cereal line
d) repeating, if necessary, steps b) and c) until an isogenic line of said second cereal, and containing said determinant, is obtained,
e) optionally, performing a self-pollination in order to obtain a cereal that is homozygous for said determinant.

The invention also relates to a method for increasing a cereal yield for a cereal harvest, comprising the step of sowing cereal seeds, wherein said cereal seeds grow into plants that exhibits an inhibited expression of an arf2 gene, and wherein the yield of the harvested cereals is increased as compared to the yield obtained from harvested isogenic cereals which do not exhibit said inhibited expression of arf2 gene.

If necessary, the comparison may be performed as mentioned above.

The invention also relates to a method of growing cereals, comprising the step of sowing the cereals of the invention, and growing cereals from the sowed seeds.

The invention may also comprise the step of harvesting said cereals.

The invention also relates to a method for harvesting cereals comprising the step of harvesting cereals of the invention.

It is clear that the cereals used in the methods described below are cereals according to the invention, in which the gene arf2 (or the production of the Arf2 protein) is inhibited, either totally or partially, in all cells or in specific tissues, totally in some tissues whereas not at all or only partially in other tissues, or with the same level of inhibition in all tissues, as described above. It is also clear that all teachings and embodiments are applicable to a cereal which presents inhibition of the expression of the arf13 gene in addition to the inhibition of the arf2 gene

### FIGURES

FIG. 1: schematic description of plasmid pBIOS1375, used to trigger RNAi in maize. The two inverted and complementary arf2 fragments needed for RNAi are indicated (ZmARF2-like partial) and are separated by a tubulin intron from rice. This RNAi fragment is driven by the CsVMV promoter.

RB, LB: Right Border, Left Border; OsTUBL intron: Tubulin intron from rice; pro Actin: rice actin promoter; +1: start of open reading frame; 3′Nos: terminator; cos Lambda: zone of recombinaison used for making the plasmid; 3′AtSac66: terminator

FIG. 2: relative level of mRNA production from T0 plants, normalized with regards to the mean of mRNA levels for EF1α and ubiquitin (see example 2). The transformation events chosen for field trial are indicated by an arrow.

FIG. 3: Table summarizing the field trial results for various transformation events (see example 3).

### EXAMPLES

### Example 1

### Generation of Vectors Intended to Perform RNAi on the Arf29 Gene in Maize, with pCSVMV

### Cloning.

### Step 1:

The ZmARF2 like fragment (SEQ ID NO 12) was amplified from genomic DNA from leaf tissue of the inbred line J1A (A188) by PCR using the following primers,

### OI 1545 ZmARF2_OI5: CACATCAAAATTCGGTCTTGCAAA (SEQ ID NO 19)

### OI 1546 ZmARF2_OI6: TTCCAGCTGTGCTTACGGACA (SEQ ID NO 20)

and then subcloned into pGEMT-Easy (Promega) forming pBIOS1351.

### Step 2:

The SpeI blunt/NotI fragment (A188 partial ZmARF2-like) from pBIOS1351 was introduced into pENTR1A (Invitrogen) opened with XmnI and NotI forming pBIOS1363 “ARF2-like Entry clone”.

### Step 3:

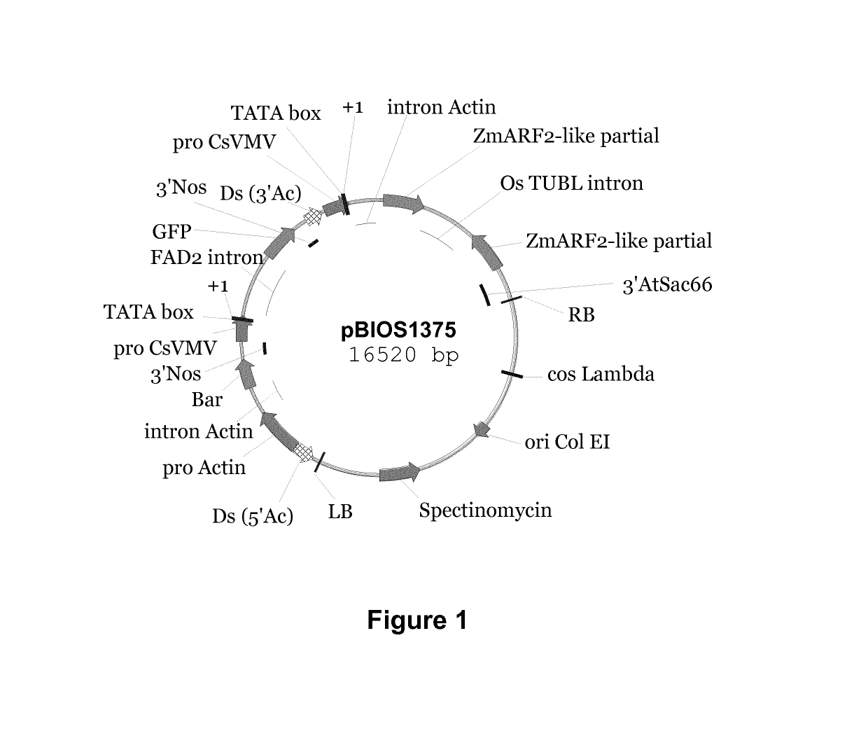

the binary plasmid pBIOS1375 (FIG. 1) was obtained by GATEWAY “LR Reaction” between pBIOS1363 “ARF2-like Entry clone” and pBIOS 893 the “Destination vector”. pBIOS 893 is a derivative of pSB11 (Komari et al (1996), Plant J. 10, 165-74) containing a pActin-Bar gene for selection of maize transformants.

pBIOS1375 was used for maize transformation according to example 2.

### Example 2

### Transformation and Characterization of Transformed Plants with Regards to the Arf29 Expression

Transformation of Maize Plants with Agrobacterium tumefaciens Carrying the RNAi Construct pBIOS1375

The transformation is effected via the transformation protocol of Ishida et al. (Nature Biotechnology, 14, 745-750, 1996) for transformation with A. tumefaciens.

Immature cobs of the A188 maize line produced under glass are taken 10 days after pollination and sterilized for 15 mins. The embryos are taken and placed in contact with a suspension of Agrobacterium containing the above described pBIOS1375 vector for 5 mins. After removal from the suspension of Agrobacterium, the embryos are cultured on a medium containing neither bacteriostatic nor selective agent. This co-culturing takes place in the dark for 4 to 7 days. After the co-culturing, the embryos are pricked out again on a fresh callogenesis medium containing the bacteriostatic and the selective agent. A callus initiates and develops from the transformed cells of these embryos. The callogenesis step takes place at 25° C. in the dark and lasts 5 weeks. The callus embryos are picked out again on fresh medium every 2 to 3 weeks. At the end of this stage, the calluses are pricked out again on a regeneration medium for 5 weeks with repicking of the callus onto fresh medium after 2 to 3 weeks, Regeneration of plantlets is effected from the calluses, which are subjected to rooting in tubes once they are sufficiently developed. After 10-15 days in tube, the plantlets are acclimatized in a phytotron before being transferred to the greenhouse.

### Preparation of RNA

Total RNA of the different samples was extracted from leaves of transformants at the T0 stage using the SV96 Total RNA Isolation System Protocol, according to the recommendations of the manufacturer (Promega).
To check the quality of RNA, a measure of Optical Density (OD) was conducted by spectrophotometry to estimate the amount and the purity of the obtained RNA, by measurement of the ratio of 260/280 (which must be higher than 1.8). An aliquot of RNA was used to verify the absence of contamination in DNA by quantitative PCR. In addition the RNA integrity was verified by capillary electrophoresis LabChip GXII (Caliper).
cDNA Synthesis
500 ng of RNA are reverse-transcribed with random hexamers in a final volume of 20 μl according to the High Capacity cDNA archive protocol by following the recommendations of the supplier (Applied Biosystems).

### Primer Design

Primer design was performed on the Arf2 sequence (GenBank accession number: BT067605) with the Primer Express v2.0 Software (Applied Biosystems) outside the area targeted by RNAi, and being as far as possible in the 3′ region. Arf2 gene was amplified with the forward primer 5′-AGCCTTCTACTATCCCGCGG-3′ (SEQ ID NO 21) and reverse primer 5′-AGGCTCTATCTTCCAGGGAGAGAC-3′ (SEQ ID NO 22) on a range of 5 points of 10 times dilution of cDNA of A188 untransformed plants to calculate the PCR efficiency.
The alignment of these primers with different members of the Arf family shows that they are not specific of Arf 29, Arf 13 is therefore also amplified in the PCR.

EF1α and Ubiquitin 2 genes are used for normalization (EF1α: forward primer 5′-TGGTGAAGATGATACCGACCAA-3′ (SEQ ID NO 23), reverse primer 5′-AGCAACCGTCTGCCTCATGT-3′ (SEQ ID NO 24), Ubiquitin 2: forward primer 5′-TGCTGGACCTCGATTGGTTT-3′ (SEQ ID NO 25), reverse primer 5′-GTCCTGCATCAACCGCTTG-3′ (SEQ ID NO 26)).

### Real-Time Quantitative PCR and Data Analysis

2 μl of a 12 times dilution of the cDNA are mixed with 3 μl primer of forward and reverse primers (1 μM each) and with 5 μl of the mix PCR containing sybrgreen Fast Start SYBR Green Master with ROX (Roche).
PCR is carried out in a volume of 10 μl final on a LightCycler 480 (Roche) at 95° C. for 5 min followed by 40 cycles at 95° C. for 15 seconds and 60° C. for 45 sec and followed by a curve of fusion according to the recommendations of the manufacturer. The threshold Cycle (Cp) is determined in an automatic way by the software of the LC480 according to the method of calculation of the maximum of the second derivative.
The data are then analyzed in Excel®:
calculation of the average of the normalization genes for each sample
calculation of the dCp=Cp ARF2−average Cp for normalization genes for each sample
calculation of the value for the relative level of expression (2^{-dCp}) for each sample
   A PCR on a non-transgenic sample (A188) was conducted to estimate the basal level of expression of the Arf2 gene. The value for the relative level of expression (2^{-dCp}) is 1.14. Consequently, all maize which harbors a relative level of expression lower than 1.14 presents an inhibition in the arf2 gene.

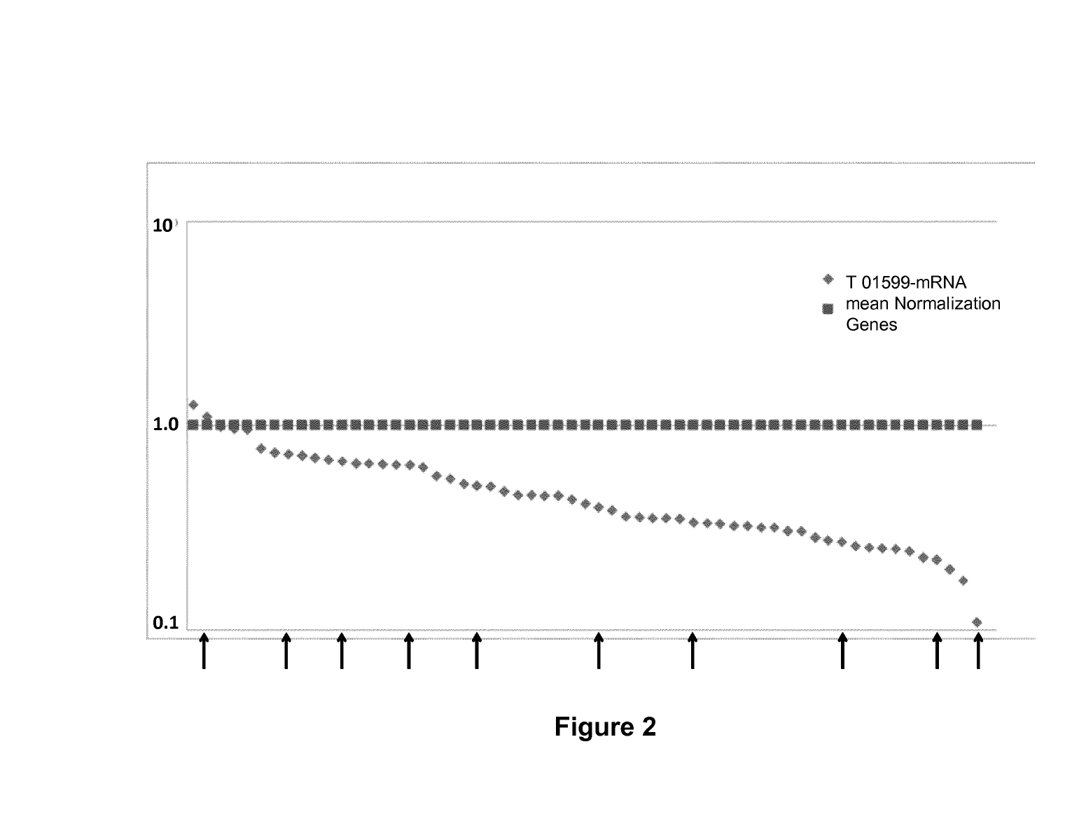

FIG. 2 describes the results obtained from different transformation events.

It can be seen that the level of expression of the arf2 gene varies depending on the transformation events. This is not surprising as it is known that the expression of a transgene depends on multiple factors (that are not well understood), such as the localization of the transgene with the plant genome. In any case, FIG. 1 demonstrates that it is possible to obtain multiple transformants that harbour a reduction in the level of expression of arf2 RNA.
Nine lines (indicated by an arrow on FIG. 2) were chosen for further analysis and field trials, some of which presenting a high inhibition of the relative level of arf2 mRNA (as compared to the A188 control), and others having a lower inhibition (see Table I).

TABLE I
Relative level of arf2 mRNA compared to A188 for T0 plants

| | Relative level of arf2 |
|---|---|
| T0 Event | mRNA compared to A188 |
| T 01599_027 | 0.19 |
| T 01599_044 | 0.29 |
| T 01599_048 | 0.63 |
| T 01599_016 | 0.56 |
| T 01599_024 | 0.44 |
| T 01599_051 | 0.24 |
| T 01599_033 | 0.10 |
| T 01599_018 | 0.58 |
| T 01599_020 | 0.96 |

### Example 3

### Field Trials

Hybrids with a tester line were obtained from T3 plants issued from the nine T0 ARF2 RNAi transgenics maize lines (CsVMV+ActInt+ARF2 RNAi+Sac66 term) chosen according to example 2.

The transformants (T0) plants were first crossed with the A188 line thereby producing T1 plants. T1 plants were then self pollinated twice, producing T3 plants which are homozygous lines containing the transgene. These T3 plants were then crossed with the tester line thereby leading to an hybrid. This hybrid is at a T4 level with regards to the transformation step and is heterozygous for the transgene. These hybrid plants are used in field experiments.

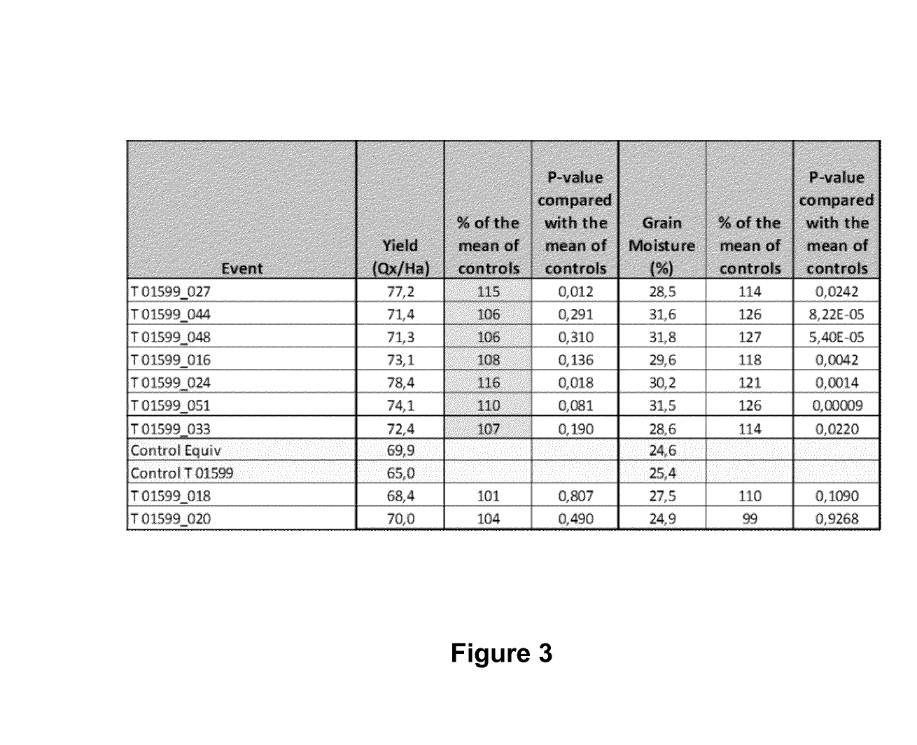

Control hybrids are obtained as follows:
Control Equiv corresponds to a cross between A188 line (the line used for transformation) and the tester line.
Control T 01599 corresponds to a cross between a null segregant (isolated after the second self-pollination of the T1 plants) and the tester line. Said null segregant is an homozygous line which does not bear the transgene. Although the null segregant theorically presents the same genome as A188, it has undergone in vitro culture (via the steps of callus differentiation and regeneration) and may thus present mutations (either genetic or epigenetic) with regards to a A188 line that has not undergone in vitro culture.
These two control lines are used to avoid any effect that could be due to mutations (genetic or epigenetic) coming from in vitro culture.
Improved yield was observed for the hybrid plants containing the RNAi construct as compared to the controls, as can be seen in FIG. 3.
It can also be seen that moisture is increased in RNAi transformed plants as compared to the controls.
Yield was calculated as follows:
During harvest, grain weight and grain moisture are measured using an on-board equipment on the combine harvester.
Grain weight is then normalized to moisture at 15%, using the following formula:

Normalize grain weight=measure grain weight×(100−measured moisture(as a percentage))/85(which is 100−normalized moisture at 15%).

As an example, if the measured grain moisture is 25%, the normalized grain weight will be: normalized grain weight=measured grain weight×75/85.
Yield is then expressed in a conventional unit (such as quintal per hectare).

### Experimental Design

Plants were sown on May 18, 2010, and harvested On Sep. 29, 2010.
The experimental block comprised 5 replicates. Each replicate comprised about 58.5 plants per plot at a density of 69600 plants/ha.
Two controls were used present in this experiment as described above (a null segregant T01599 and a control equivalent (A188 crossed with the tester line).
Results are represented on FIG. 3, with the yield expressed in (Qx/ha).
This figure demonstrates that the transgenic plants present an increased yield (normalized for moisture) and an increased grain moisture compared to the control. No sterility abnormalities such as the ones described for the arl2-mutant, RNAi or co-suppressed Arabidopsis of the prior art were observed for these plants.

### Example 4

Other RNAi constructs can be designed for inhibition of the arf2 gene in wheat, rice, sorghum or brachypodium, using respectively the sequences SEQ ID NO 13, SEQ ID NO 14, SEQ ID NO 15, SEQ ID NO 16 as sequences for the “sense” part of the RNAi construct.

1. A cereal comprising at least one cell which presents inhibition of Arf2 expression, by inhibition of the arf2 gene coding for the Arf2 protein, wherein alleles of said Arf2 protein are depicted by SEQ ID NO 1 (for maize), SEQ ID NO 3 (for rice), SEQ ID NO 4 or SEQ ID NO 27 (for barley), SEQ ID NO 5 (for sorghum), SEQ ID NO 6 (for Brachypodium) and SEQ ID NO 7 to SEQ ID NO 9 (corresponding to the Arf2 proteins for the three genomes of wheat).

2. The cereal of claim 1, which is maize, and wherein an allele of said Arf2 protein exhibit SEQ ID NO 1.

3. The cereal of claim 1, wherein said Arf2-coding gene is inhibited in specific tissue(s) of said cereal and is not inhibited in other tissues of said cereal.

4. The cereal of claim 1, wherein said inhibition of said Arf2-coding gene is a partial inhibition.

5. The cereal of claim 1, wherein said inhibition of said Arf2-coding gene is a total (full) inhibition.

6. The cereal of claim 1, wherein inhibition of the Arf2 expression is due to a mutation of the arf2 gene through insertion of a transposable element or of a T-DNA or following physical mutagenesis.

7. The cereal of claim 1, wherein inhibition of the Arf2 expression is due to the presence, in the genome of said cereal of an antisense, or overexpression (leading to co-suppression), or RNAi construct.

8. The cereal of claim 7, wherein inhibition of the Arf2 expression is due to the presence, in the genome of said cereal, of a RNAi construct, wherein said construct is under the control of a constitutive promoter.

9. The cereal of claim 8, wherein said promoter is pCSVMV (represented by SEQ ID NO 17).

10. The cereal of claim 4, wherein said construct comprises a RNAi construct, the sequence of which is depicted by SEQ ID NO 18.

11. A method for improving yield in a cereal, comprising inhibiting the expression of the arf2 gene coding for the Arf2 protein in said cereal, wherein said cereal with the inhibited arf2 gene presents a higher yield than a cereal not having an inhibited arf2 gene.

12. A method for producing a cereal that can be used in a selection process for obtaining a cereal with improved yield, comprising the step of inhibiting the expression of the arf2 gene coding for the Arf2 protein in said cereal.

13. The method of claim 12, wherein said inhibition is obtained by transformation of said cereal with an antisense, or overexpression, or RNAi construct, or by mutation of the arf2 gene, through insertion of a transposable element or of a T-DNA or by physical mutagenesis.

14. A method for increasing cereal yield, comprising the step of sowing cereal seeds, wherein said cereal seeds grow into plants that exhibits an inhibited expression of the arf2 gene coding for the Arf2 protein, and wherein the yield of the harvested cereals is increased as compared to the yield obtained from harvested cereals not exhibiting an inhibited expression of the arf2 gene.

15. A method for selecting a cereal that can be used in a selection process for obtaining a cereal with improved yield comprising the step of selecting, in a population of cereals, the cereals in which the arf2 gene is inhibited.
